(19) United States
(12) Patent Application Publication
(10) Pub. No.: US 2004/0219566 A1
(43) Pub. Date: Nov. 4, 2004

(54) cDNA microarray data correction system, method, program, and memory medium

(75) Inventors: Masataka Andoh, Tokyo (JP); Akira Saitoh, Tokyo (JP); Megu Ohtaki, Hiroshima (JP); Kenichi Satoh, Hiroshima (JP); Masahiko Nishiyama, Hiroshima (JP); Keiko Otani, Tokyo (JP)

Correspondence Address: FOLEY AND LARDNER, SUITE 500, 3000 K STREET NW, WASHINGTON, DC, 20007, US

(73) Assignees: NEC CORPORATION; MEGU OHTAKI; JAPAN BIOLOGICAL INFORMATICS CONSORTIUM

(21) Appl. No.: 10/696,572

(22) Filed: Oct. 30, 2003

(30) Foreign Application Priority Data
Apr. 28, 2003 (JP) 2003-124585

(51) Int. Cl. C12Q 1/68; G06F 19/00; G01N 33/48; G01N 33/50

(52) U.S. Cl. 435/6; 702/20

(57) ABSTRACT

More precise correction of global and local distortions of microarray data and correction of measurement errors caused by a difference in sensitivity between fluorescent dyes. A data standardization unit for a first process inputs gene expression intensity data from an input device, standardizes the gene expression intensity data by using grid-by-grid order statistics on the assumption that most genes are in a non-expression state, and outputs the standardized gene expression intensity data. A spot-position-based correction unit for a second process estimates a distortion depending on a spot position on a grid by grid basis by a nonparametric smoothing method and outputs gene expression intensity data whose distortion depending on the spot position has been corrected. An S-D-plot-based correction unit for a third process performs an S-D transformation, estimates a distortion caused by a difference in sensitivity between the fluorescent dyes by the nonparametric smoothing method, and outputs the gene expression intensity data whose distortion caused by the difference in sensitivity between the fluorescent dyes has been corrected to the output device.

### BACKGROUND OF THE INVENTION

The present invention relates to a data correction system, method, program, and memory medium for cDNA microarray data based on a mathematical model, and more particularly to a cDNA microarray data correction system, method, program, and memory medium enabling global normalization and local normalization and further enabling a correction of a distortion of measurements caused by a difference in sensitivity between fluorescent dyes.

### RELATED BACKGROUND ART

The genome research is developing from an individual gene structural analysis into a systematic gene functional analysis at present. Experiments using complementary DNA (cDNA) microarray is greatly expected to display effectiveness due to its ability to quantify an expression intensity of a lot of genes simultaneously for a functional analysis of genes whose functions are unknown or genes in the mass.

The purpose of an experiment using the cDNA microarray in a two-color fluorescence technique is to detect a difference in gene expression between two types of cells. An outline of the cDNA microarray in the two-color fluorescence technique is then described here. First, a lot of gene sets of cDNA are d nsely fix d as a r fer nc probe in an array on a slide glass (microarray).

Subsequently, mRNAs sampled from two types of samples under different conditions, cell 1 and cell 2 (for example, a normal cell and a cancer cell) are labeled with fluorescent dyes having different wavelengths for a synthesis of a target cDNA. Then, those mixed in equal proportions are used for competitive hybridization with the reference probe cDNA fixed to the microarray. After the hybridization, the intensities of the fluorescent dyes are measured using a scanner. The fluorescent dye on the cell 1 and the fluorescent dye on the cell 2 are read in channel 1 and channel 2, respectively, and they are considered to be gene expression intensity data of the respective cells ((microarray data).

Thus, the process of achieving the microarray data is complicated and requires advanced experimental techniques. Thereby various experimental errors are anticipated in various stages of the experiment. Therefore, an analysis of a distribution of gene expression intensities and experimental errors are important problems in order to extract truly biologically significant data from the microarray data.

Regarding the distribution of gene expression intensities, for example, with reference to a document 1 (Journal of Computational Biology Vol. 8, pp. 37-52), Newton et al. considered the statistical property of a gene expression intensity ratio (ratio of gene expression intensity data between channel 1 and channel 2), assuming gamma distribution functions for gene expression intensities.

In addition, for observed gene expression intensity data, for example, with reference to a document 2 (Proceeding of the National Academy of Sciences Vol. 97, No 18, pp. 9934-9839), Lee et al. applied a mixture normal distribution as represented by the following EQ1 to the statistical consideration of the gene expression intensity data, on the assumption that true gene expression intensities can be separated into two levels and that random errors exist.

f(x)=pΦ(x−μ₁|σ₁²)+(1−p)Φ(x−μ₂|σ₂²) (1)

In the above, x indicates gene expression intensity data such as a fluorescence intensity obtained using a scanner, Φ(x−μ₁|σ₁²) in the first term of the right-hand side indicates a density function of a normal distribution of average μ₁ and variance σ₁² in a gene expression state, Φ(x−μ₁|σ₂²) in the second term of the right-hand side indicates a density function of a normal distribution of average μ₂ and variance σ₂² in a gene non-expression state, and p is a population parameter indicating their mixing rate.

Regarding the analysis of experimental errors, some methods of removing systematic errors, namely, normalization methods have been suggested. There are main two types of normalization; global normalization intended for all spots on an array and local standardization intended for spots in units of a subset (for example, in units of a grid). Regarding the global normalization, for example, with reference to a document 3 (Journal of Biomedical Optics Vol. 2, pp. 364-374), Chen et al. corrected measurements obtained in channel 1 and channel 2 assuming that medians of gene expression intensities of two cells are equal. Regarding the local normalization, for example, with reference to a documents 4 (Dudoit et. al, 2000. Statistical methods for identifying differentially expressed genes in replicated cDNA microarray experiments. Technical-Report #578 2.), 5(Nucleic Acids Research, 2000, Vol.28, No. 10), and 6(Nucleic Acids Research, 2002, Vol.30, No. 4), Dudoit, Shuchhardt, and Yang thought that systematic errors are caused by differences in a spot position on a slide glass or in sensitivity between two types of fluorescent dyes and suggested a method for removing them.

### SUMMARY OF THE INVENTION

A problem in the conventional technologies in the above is that the microarray data lacks in reproducibility with a tendency to be unstable and thereby its precision or efficiency is considered to be low. It is because true signals on the gene expression are not fully separated from experimental errors. As a factor behind it, it is attributed to the fact that the gene expression intensities could have various levels depending on the gene. If so, the above model represented by EQ1 is apparently too simplified.

It is an object of the present invention to provide a comprehensive normalization method and system for correcting global and local distortions with high precision and further correcting measurement errors caused by a difference in sensitivity between fluorescent dyes by assuming more accountable mathematical model of gene expression intensity data on a microarray.

In accordance with an aspect of the present invention, there is provided a cDNA microarray data correction system, comprising an input device for inputting gene expression intensity data such as a fluorescence intensity, a data analyzer operating with program controls, and an output device. It is assumed that the gene expression intensity data to be input is previously adjusted, considering flag information indicating a removal of background noise of each spot and reliability of the spot.

The data analyzer has the following three continuous processes. A data standardization unit for a first process inputs gene expression intensity data from the input device, standardizes the gene expression intensity data by using grid-by-grid order statistics on the assumption that most genes are in a non-expression state, and outputs the standardized gene expression intensity data.

A spot-position-based correction unit for a second process inputs the standardized gene expression intensity data, estimates a distortion depending on the spot position on a grid by grid basis by a nonparametric smoothing method, and outputs gene expression intensity data whose distortion depending on the spot position has been corrected.

An S-D-plot-based correction unit for a third process performs an S-D transformation, which is a variation of an MA transformation (for information about the MA transformation and an MA plot, refer to the above nonpatent document 6), for the gene expression intensity data corrected up to the second process, estimates a potential distortion caused by a difference in sensitivity between the fluorescent dyes in the gene expression intensity data by the nonparametric smoothing method, and outputs the gene expression intensity data whose distortion caused by the difference in sensitivity between the fluorescent dyes has been corrected to the output device.

This system further comprises an S-D transformation unit for quantifying the distortion of the gene expression intensity data in an arbitrary stage and for visualizing it on the S-D plot.

By using the constitution to correct the gene expression intensity data, the object of the present invention can be achieved.

### BRIEF DESCRIPTION OF THE DRAWINGS

FIG. 1 is a diagram showing a microarray structure according to the present invention;

FIG. 2 is a block diagram showing a configuration of a first embodiment of the present invention;

FIG. 3 is a flowchart showing an operation of the first embodiment of the present invention;

FIG. 4 is a block diagram showing a configuration of a second embodiment of the present invention;

FIG. 5 is a diagram showing gene expression intensities of original data obtained in channel 1;

FIG. 6 is a diagram showing gene expression intensities of original data obtained in channel 2;

FIG. 7 is a diagram showing gene expression intensities of original data (a first grid to a fourth grid) obtained in the channel 1;

FIG. 8 is a diagram showing gene expression intensities of original data (a first grid to a fourth grid) obtained in the channel 2;

FIG. 9 is an S-D plot to the original data;

FIG. 10 is a diagram showing gene expression intensities of the original data in the channel 1;

FIG. 11 is a diagram showing gene expression intensities after a first process in the channel 1;

FIG. 12 is a diagram showing gene expression intensities after a second process in the channel 1;

FIG. 13 is a diagram showing gene expression intensities after a third process in the channel 1;

FIG. 14 is a diagram showing gene expression intensities of the original data in the channel 2;

FIG. 15 is a diagram showing gene expression intensities after the first process in the channel 2;

FIG. 16 is a diagram showing gene expression intensities after the second process in the channel 2;

FIG. 17 is a diagram showing gene expression intensities after the third process in the channel 2;

FIG. 18 is an S-D plot to the original data;

FIG. 19 is an S-D plot after the first process;

FIG. 20 is an S-D plot after the second process; and

FIG. 21 is an S-D plot after the third process.

### DESCRIPTION OF THE PREFERRED EMBODIMENTS

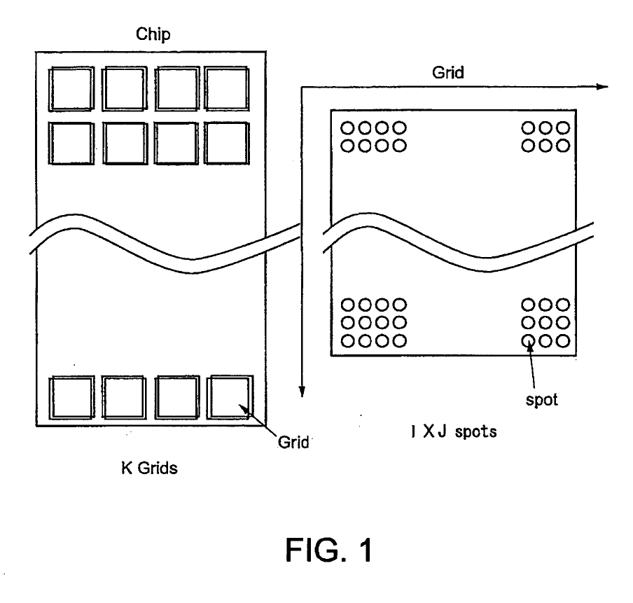

First, a microarray structure in the present invention will be described hereinafter. Referring to FIG. 1, there are cDNA spots on a slide glass having K grids by I×J spots per grid, that is, K×I×J spots in total. In this condition, it is assumed that yᵢⱼ^{k}(c), c=1, 2 is a fluorescence intensity obtained in channel c (c=1, 2) for the cDNA spots on the coordinates (i, j) in grid k.

Subsequently, the following two assumptions are provided.

Supposing that a probability of gene expression is lower than 0.5, it is assumed that the fluorescence intensity yᵢⱼ^{k}(c) detected at more than half of the spots within each grid indicates a background noise or a systematic error (Assumption 1).

Supposing that Lₖ(c) and Mₖ(c) indicate 25 and 50 percent points of the fluorescence intensity yᵢⱼ^{k}(c) obtained in the channel c in the grid k, then it is assumed that Lₖ(c) and Mₖ(c)−Lₖ(c) are common to all grids and channels on condition that most genes are in a non-expression state and that the distribution under 50 percent point of the fluorescence intensity is common to all grids and channels (Assumption 2).

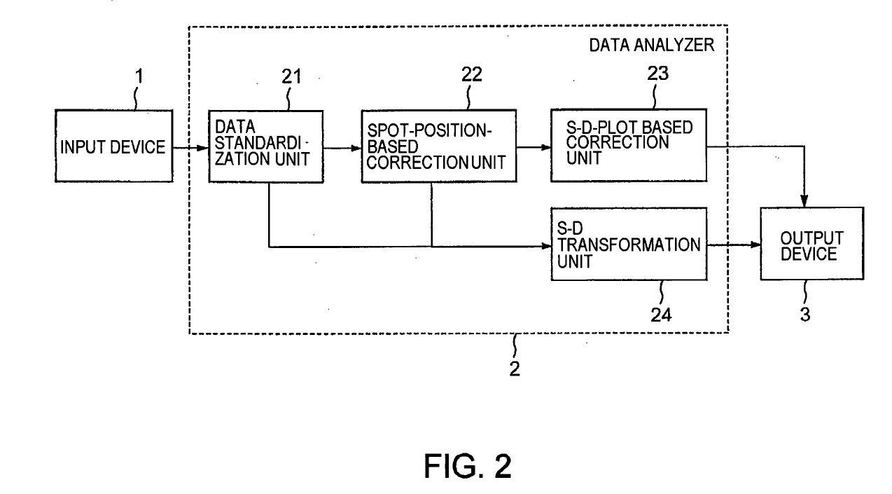

Subsequently, based on the above assumptions, the first embodiment of the present invention will be described in detail by referring to appended drawings. Referring to FIG. 2, there is shown the first embodiment of the present invention comprising an input device 1 for inputting gene expression intensity data such as a fluorescence intensity, a data analyzer 2 operating with program controls, and an output device 3 such as a display unit or a printer. The data analyzer includes a data standardization unit 21, a spot-position-based correction unit 22, and an S-D-plot-based correction unit 23.

The data standardization unit 21 standardizes gene expression intensity data by using grid-by-grid order statistics for given gene expression intensity data and then transmits it to the spot-position-based correction unit 22 and an S-D transformation unit 24.

The spot-position-based correction unit 22 estimates a distortion depending on the spot position on a grid by grid basis by a nonparametric smoothing method for the standardized gene expression intensity data transmitted from the data standardization unit 21 and then transmits corrected gene expression intensity data to the S-D-plot-based correction unit 23 and the S-D transformation unit 24.

The S-D-plot-based correction unit 23 performs an S-D transformation for the corrected gene expression intensity data transmitted from the spot-position-based correction unit 22, corrects a distortion caused by a difference in sensitivity between fluorescent dyes by the nonparametric smoothing method, and outputs the gene expression intensity data to the output device 3.

The S-D transformation unit 24 performs an S-D transformation for the gene expression intensity data transmitted from the spot-position-based correction unit 22 and then transmits it to the output device 3.

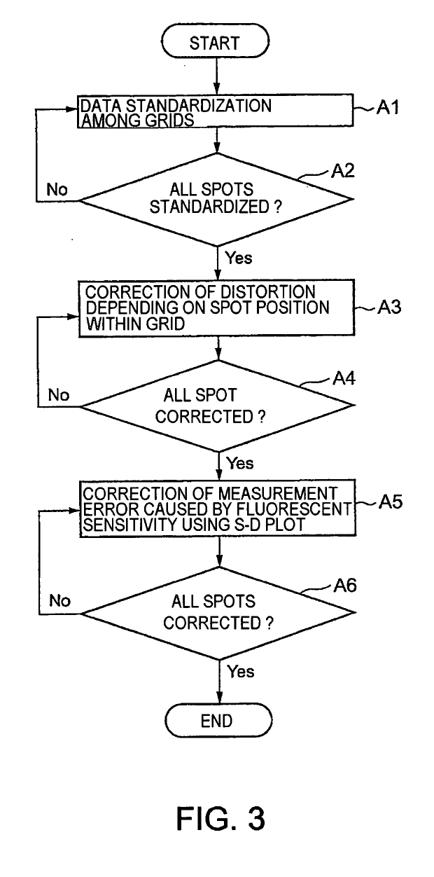

Subsequently, the embodiment will be described in detail by referring to FIG. 2 and FIG. 3. The gene expression intensity data such as fluorescence intensity input from the input device 1 is transmitted to the data standardization unit 21. The data standardization unit 21 standardizes the expression intensity data it has received by using grid-by-grid order statistics as represented by the following EQ2 (step A1 in FIG. 3).

$$\begin{matrix}{w}_{i j}^{k} ( c ) = \frac{{y}_{i j}^{k} ( c ) - {L}_{k} ( c )}{{M}_{k} ( c ) - {L}_{k} ( c )} , \\ c = 1 , 2 , i = 1 , … , I , j = 1 , … , J , k = 1 , … , K .\end{matrix} \qquad ( 2 )$$

It is determined whether the gene expression intensity data yᵢⱼ^{k}(c) of all spots obtained in the two channels have been standardized. This operation is continued until the standardization of the gene expression intensity data (2×I×J×K pieces) of all spots is completed (step A2).

For the gene expression intensity data wᵢⱼ^{k}(c) standardized by the data standardization unit 21, it is assumed that zᵢⱼ^{k}(c) is a fluorescence intensity reflecting a true expression intensity (hereinafter, referred to as a true expression fluorescence intensity) and that ξᵢⱼ^{k}(c) is a spot-position-dependent distortion on coordinates (i, j) of the grid k. In this condition, it is assumed that gene expression intensity data wᵢⱼ^{k}(c) is represented by a sun of the true expression intensity zᵢⱼ^{k}(c) and the spot-position-dependent distortion ξᵢⱼ^{k}(c), as represented by the following EQ3.

wᵢⱼ^{k}(c)=zᵢⱼ^{k}(c)+ξᵢⱼ^{k}(c), εᵢⱼ^{k}(c), εᵢⱼ^{k}(c)˜N(0, σₖ(c)²), c=1,2 (3)

In the above, εᵢⱼ^{k}(c) is assumed a random noise.

The spot-position-based correction unit 22 describes the spot-position-dependent distortion ξᵢⱼ^{k}(c) by means of a nonparametric regression model represented by a regression relation of distortions with an x-axis, a y-axis, and an interaction of the two axes as represented by the following EQ4 and estimates the spot-position-dependent distortion ξᵢⱼ^{k}(c) by using the nonparametric smoothing method as represented by the following EQ5.

$$\begin{matrix}{ξ}_{i j}^{k} ( c ) = {α}_{k}^{( C )} ( i ) + {β}_{k}^{( C )} ( j ) + {γ}_{k}^{( C )} ( ( i - {m}_{i} ) ( j - {m}_{j} ) ) , & ( 4 ) \\ c = 1 , 2 , i = 1 , … , I , j = 1 , … , J , & \\ {\sum }_{i} {α}_{k}^{( C )} ( i ) = 0 , {\sum }_{j} {β}_{k}^{( C )} ( j ) = 0 , {\sum }_{i} {\sum }_{j} {γ}_{k}^{( C )} ( ( i - {m}_{i} ) ( j - {m}_{j} ) ) = 0. & \\ {\hat{ξ}}_{i j}^{k} ( c ) = {\hat{α}}_{k}^{( C )} ( i ) + {\hat{β}}_{k}^{( C )} ( j ) + {\hat{γ}}_{k}^{( C )} ( ( i - {m}_{i} ) ( j - {m}_{j} ) ) , & ( 5 ) \\ c = 1 , 2 , i = 1 , … , I , j = 1 , … , J . & \end{matrix}$$

In the above, mᵢ=└I/2┘, mⱼ=└J/2┘ is assumed. └α┘ is assumed to be the minimum integer equal to or more than α.

The spot-position-based correction unit 22 corrects the estimated spot-position-dependent distortion ξᵢⱼ^{k}(c) for the gene expression intensity data wᵢⱼ^{k}(c) standardized by the data standardization unit 21 (step A3) as represented by the following EQ6.

\hat{z}ᵢⱼ^{k}(c)=wᵢⱼ^{k}(c)−\hat{ξ}ᵢⱼ^{k}(c) (6)

It is determined whether the spot-position-dependent distortion \hat{ξ}ᵢⱼ^{k}(c) has been corrected for the gene expression intensity data wᵢⱼ^{k}(c) of all spots standardized by the data standardization unit 21. This operation is continued until the correction is completed for the gene expression intensity data (2×I×J×K pieces) of all spots (step A4).

The S-D-plot-based correction unit 23 performs the S-D transformation for the true gene expression intensity data \hat{z}ᵢⱼ^{k}(c) corrected by the spot-position-based correction unit 22 as represented by the following EQ7.

$$\begin{matrix}{u}_{i j}^{k} = {\overline{z}}_{i j}^{k} ( 1 ) + {\overline{z}}_{i j}^{k} ( 2 ) \\ {v}_{i j}^{k} = {\hat{z}}_{i j}^{k} ( 1 ) - {\hat{z}}_{i j}^{k} ( 2 )\end{matrix} \qquad ( 7 )$$

Furthermore, with a description of a nonparametric regression model as represented by the following EQ8, a measurement error caused by a difference in sensitivity between the fluorescent dyes is corrected after estimating it by the nonparametric smoothing method as represented by the following EQ9 and EQ10 (step A5).

vᵢⱼ^{k}=φ(uᵢⱼ^{k})+εᵢⱼ^{k}, εᵢⱼ^{k}=N(0, v²) (8)

ηᵢⱼ^{k}=vᵢⱼ^{k}−\hat{φ}(uᵢⱼ^{k}) (9)

$$\begin{matrix}{\hat{y}}_{i j}^{k} ( 1 ) = \frac{1}{2} ( {u}_{i j}^{k} + {η}_{i j}^{k} ) \\ {\overline{y}}_{i j}^{k} ( 2 ) = \frac{1}{2} ( {u}_{i j}^{k} - {η}_{i j}^{k} )\end{matrix} \qquad ( 10 )$$

It is determined whether the correction with the S-D plot has been performed for the true gene expression intensity data \hat{z}ᵢⱼ^{k}(c) corrected by the spot-position-based correction unit 22. This operation is continued until the correction is completed for the true gene expression intensity data (2×I×J×K pieces) of all spots (step A6).

After a completion of the steps A2 and A4 in FIG. 3, the gene expression intensity data is transmitted to the output device 3 via the S-D transformation unit 24, by which the distortion of the gene expression intensity data can be visualized by the S-D plot,

Subsequently, effects of the embodiment will be described below. In the embodiment, the standardization has been made by combining standardization using order statistics over the grids (global standardization) and the correction of a distortion depending on the spot position within a grid (local standardization). Thereby, it becomes possible to correct a systematic error caused by deviation of the gene expression intensities among the grids and a distortion depending on the spot position within an individual grid at a time. Furthermore, in the correction with the S-D plot, the measurement error caused by a difference in sensitivity between fluorescent dyes can be corrected by using a sum and a difference of the expression intensity data.

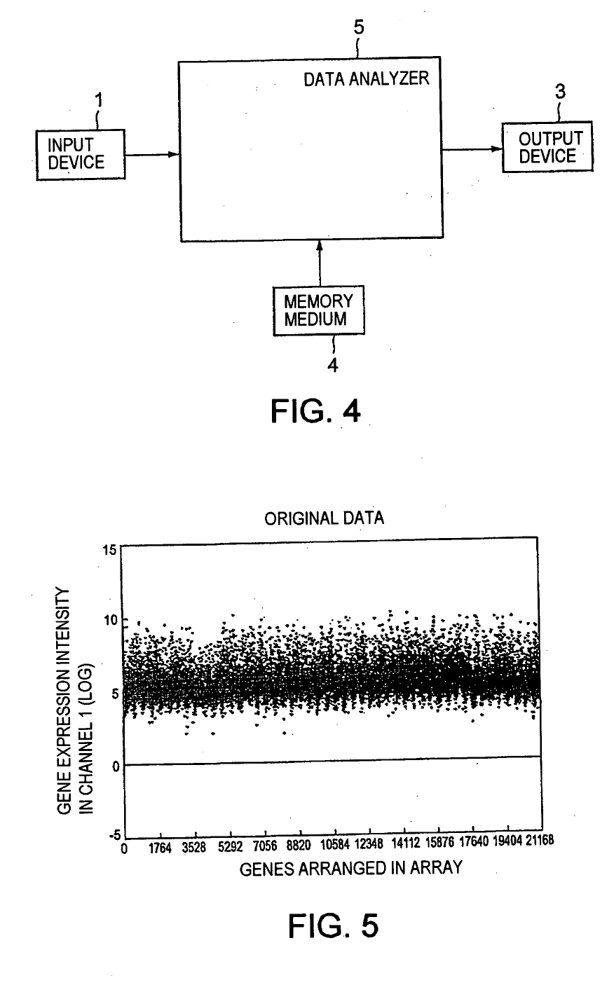

A second embodiment of the present invention will now be described in detail hereinafter by referring to appended drawings. Referring to FIG. 4, there is shown the second embodiment of the present invention comprising an input device, a data analyzer, and an output device similarly to the first embodiment, and further comprising a memory medium 4 where a data analysis program is recorded. The memory medium 4 can be either transportable or of stationary type, and can be a magnetic disk, a semiconductor memory, a CD-ROM, or any other memory medium.

In addition, it is also possible to previously store a computer program capable of executing the method of the present invention in a recording device of a computer connected to a network and to transfer the program to another computer via the network. A medium for providing the computer program for executing this algorithm can be distributed as a medium whose data can be read out to computers in various formats, and it is not limited to a specific type of mediums. The data analysis program is read from the memory medium 4 to a data analyzer 5 to control operations of the data analyzer 5, thereby executing the same processing as in the data analyzer 2 of the first embodiment for the data file input from the input device 1.

The embodiment of the present invention will be described hereinafter. Data for the example is obtained from an experiment for a comparison between two different types of cancer cells (cell A and cell B) in the gene expression condition.

The following is a result of checking gene expression patterns in 48 grids on a single chip with 441 (21×21) spots per grid, namely, 21,168 spots in total.

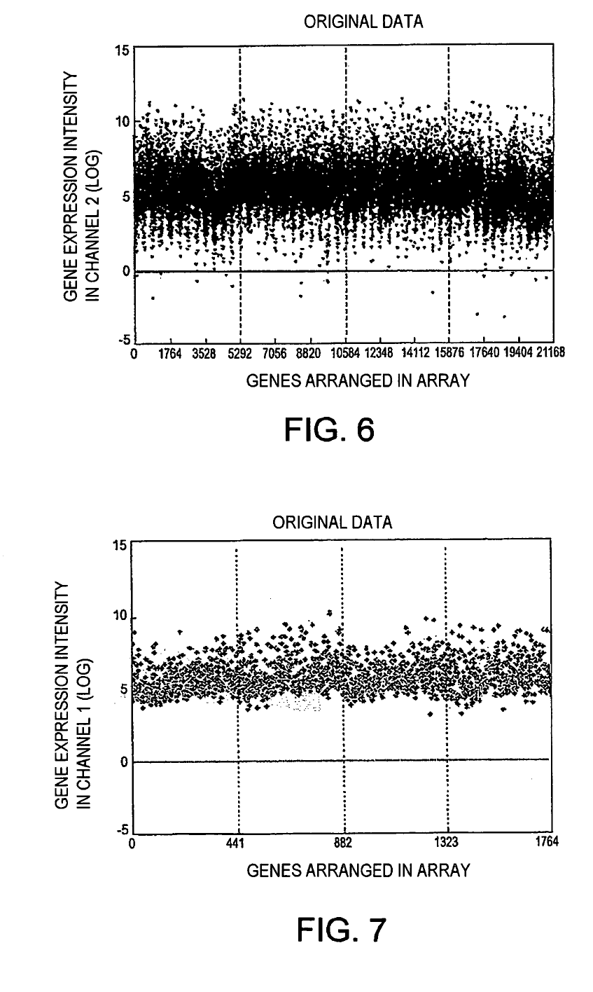

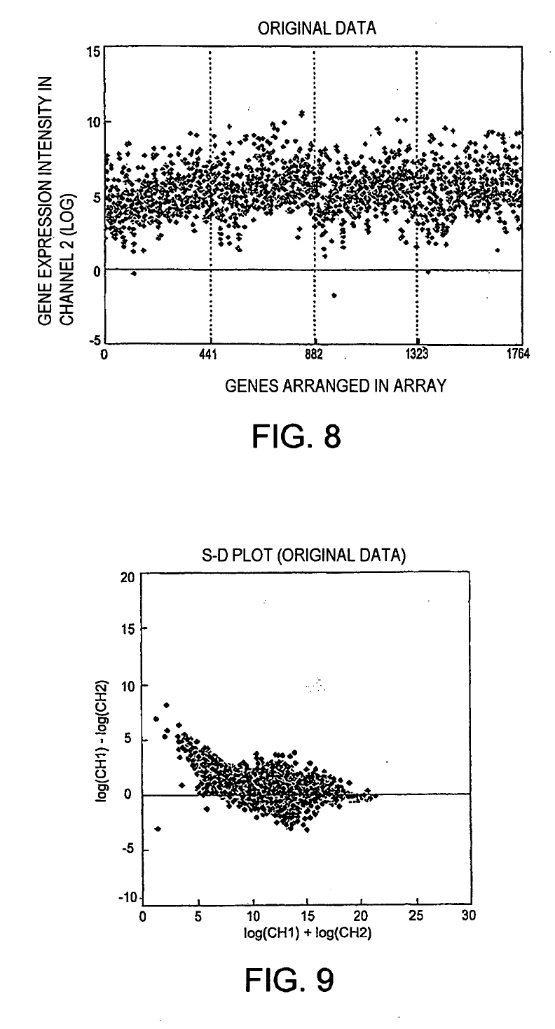

Referring to FIG. 5 and FIG. 7, there are shown gene expression intensities of the cell A of original data obtained in channel 1. Referring to FIG. 6 and FIG. 8, there are shown gene expression intensities of the cell B of original data obtained in channel 2. These graphs show plotting of logarithmic values of the gene expression intensities of the spot positions on the microarray. FIG. 7 and FIG. 8 are enlarged views of the first to fourth grids. As is shown by FIG. 5 to FIG. 8, we can observe a systematic distortion periodically repeated in gene expression intensities on a grid by grid basis. Since the gene spots are arranged at random on the microarray, the distortion is thought to be an experimental error.

Referring to FIG. 9, there is shown an S-D plot of the distortion. The abscissa indicates a sum of the gene expression intensities of the channels and the ordinate indicates a difference between them. In areas where the sum of the gene expression intensities of the channels is small or large, the difference of the gene expression intensities between the channels is not so much affected by a true gene expression difference. It is then thought to occur due to a difference in sensitivity between fluorescent dyes of the channels. Thereby, we can observe the distortion thought to occur due to the difference in sensitivity between the fluorescent dyes in FIG. 9.

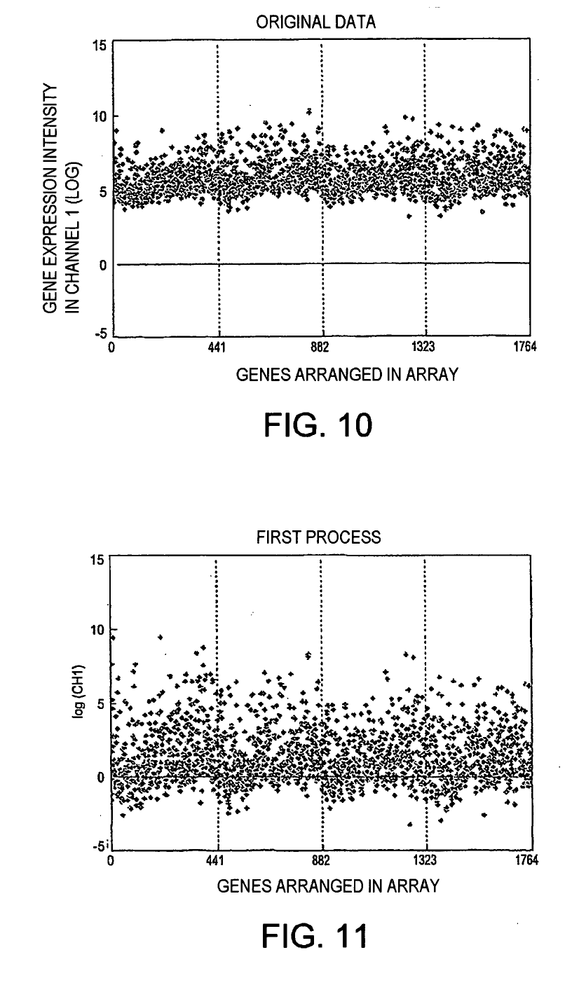

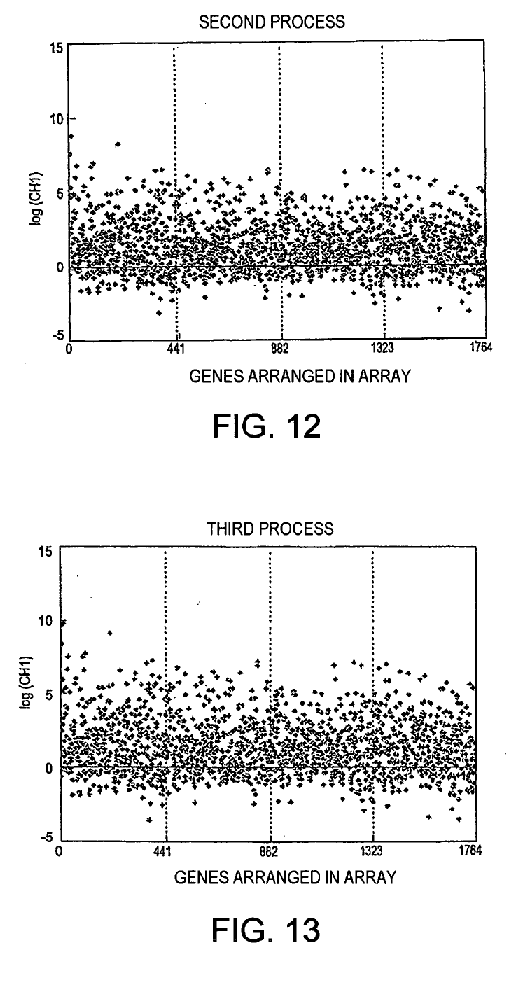

Referring to FIG. 10, there is shown a diagram of gene expression intensities of the spot positions of the original data in the channel 1. Referring to FIG. 11, there is shown a diagram of gene expression intensities of the spot positions after the first process in the channel 1. Referring to FIG. 12, there is shown a diagram of gene expression intensities of the spot positions after the second process in the channel 1. They show that the systematic distortion periodically repeated on a grid by grid basis, depending on the spot position, has been removed by correction.

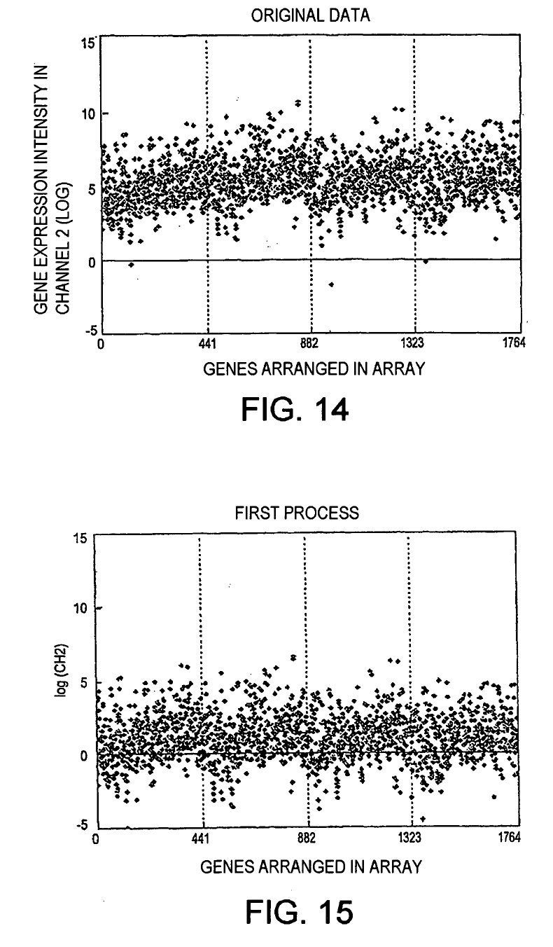

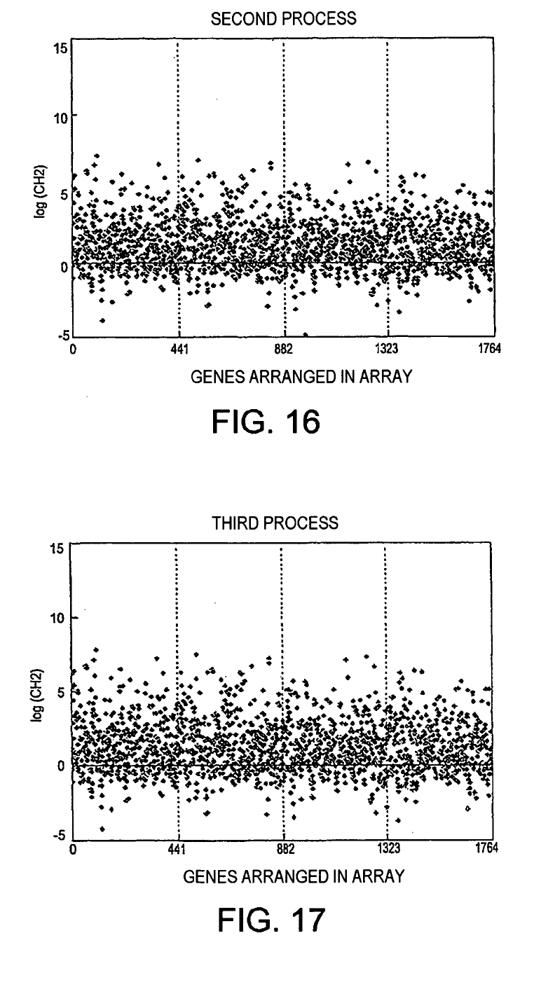

Referring to FIG. 13, there is shown a diagram of gene expression intensities of the spot positions after the third process in the channel 1. Referring to FIG. 14 to FIG. 17, there are shown diagrams of gene expression intensities of the spot positions of the original data, after the first process, after the second process, and after the third process in the channel 2. Similarly to the channel 1, they show that the systematic distortion periodically repeated on a grid by grid basis, depending on the spot position, has been removed by correction.

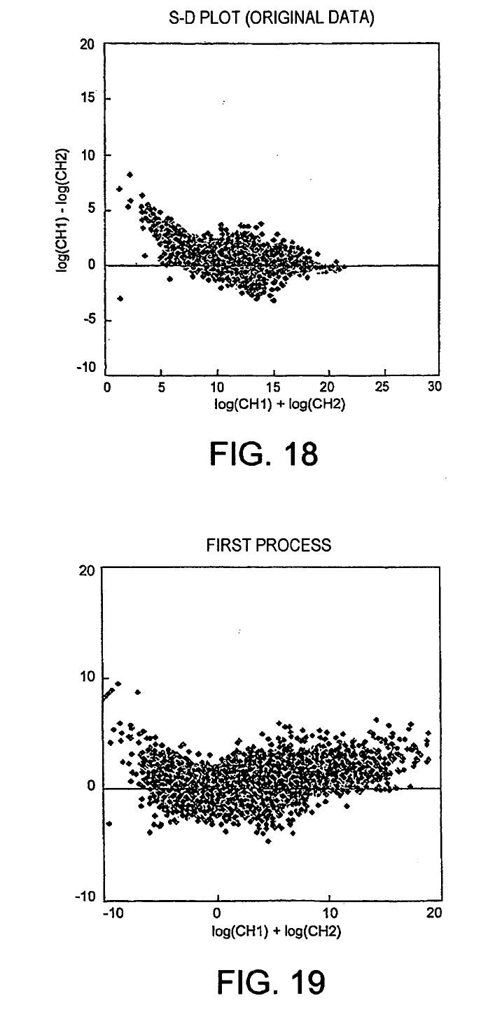

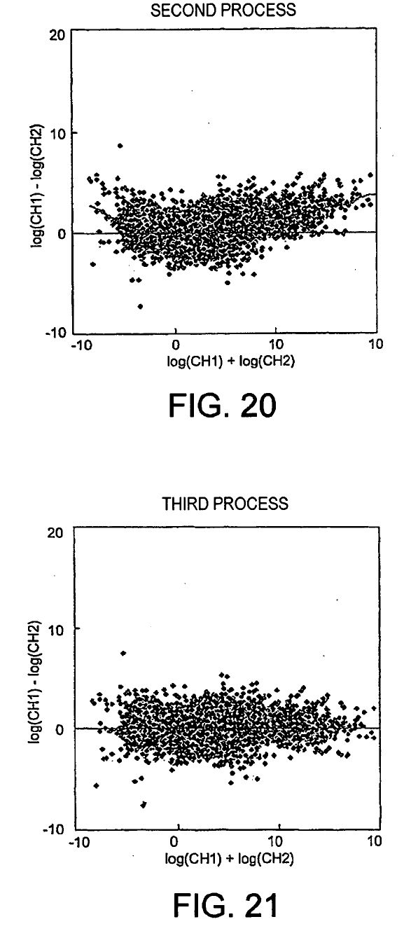

Referring to FIG. 18 to FIG. 21, there are shown S-D plots of the original data, after the first process, after the second process, and after the third process. As is shown by FIG. 21, it is apparent that the distortion caused by the difference in sensitivity between the fluorescent dyes has been removed by correction.

According to the present invention, the standardization is performed by combining the standardization with the stable order statistics, 25 and 50 percent points, for fluctuations of the position and scale among the grids (global normalization) and the correction of a distortion depending on the spot position within a grid (local normalization). Thereby, it is possible to simultaneously correct a systematic error caused by deviation of the gene expression intensities among grids or by fluctuations of sensitivity and a distortion depending on the spot position within a grid, almost without any effect of a gene expression frequency nor an outlier.

Furthermore, according to the present invention, a difference in sensitivity between the fluorescent dyes can be easily obtained by using a sum and a difference of the gene expression intensity data in the S-D plot, thus enabling an accurate extraction of a measurement error caused by the difference. Thereby, it is possible to correct efficiently a distortion of measurements caused by the difference in sensitivity between the fluorescent dyes.

Referring to Assumption 2 above, Lₖ(c) and Mₖ(c) indicate 25 and 50 percent points respectively. Supposing that Aₖ(c), Lₖ(c) and Mₖ(c) indicate 35, 10 and 90 percent points of the fluorescence intensity yᵢⱼ^{k}(c) obtained in the channel c in the grid k respectively, it is assumed that Aₖ(c) and Mₖ(c)−Lₖ(c) are more common to all grids and channels. Thereby, it is possible to effectively correct a systematic error. In this case, the data standardization unit 21 standardizes the expression intensity data it has received by using grid-by-grid order statistics as represented by the following EQ11 (global normalization) (referring to FIG. 2).

$$\begin{matrix}{w}_{i j}^{k} ( c ) = \frac{{y}_{i j}^{k} ( c ) - {A}_{k} ( c )}{{M}_{k} ( c ) - {L}_{k} ( c )} , \\ c = 1 , 2 , i = 1 , … , I , j = 1 , … , J , k = 1 , … , K .\end{matrix} \qquad ( 11 )$$

1. A cDNA microarray data correction system for correcting global and local distortions of microarray data more precisely and correcting measurement errors caused by a difference in sensitivity between fluorescent dyes, comprising:
an input device for inputting previously-adjusted gene expression intensity data, considering flag information indicating a removal of background noise and reliability of each spot;
a data standardization means for standardizing the gene expression intensity data by using grid-by-grid order statistics for the input gene expression intensity data and for transmitting the standardized gene expression intensity data;
first correction means for estimating a distortion depending on a spot position on grid coordinates for the standardized gene expression intensity data by a nonparametric smoothing method and for transmitting first corrected gene expression intensity data whose distortion has been corrected; and
second correction means for performing an S-D transformation for the first corrected gene expression intensity data, for estimating a potential distortion caused by a difference in sensitivity between the fluorescent dyes in the gene expression intensity data by the nonparametric smoothing method, and for transmitting second corrected gene expression intensity data whose distortion caused by the difference in sensitivity between the fluorescent dyes has been corrected; and
an output device for outputting the second corrected gene expression intensity data.

2. The cDNA microarray data correction system according to claim 1, further comprising S-D transformation means for quantifying the distortion of the gene expression intensity data in an arbitrary stage and for visualizing it on an S-D plot.

3. The cDNA microarray data correction system according to claim 1, wherein the order statistics are represented by the following EQ12 (where wᵢⱼ^{k}(c) is the standardized gene expression intensity data, yᵢⱼ^{k}(c) is gene expression intensity data of all spots obtained in a channel, and Lk(c) and Mk(c) indicate 25 and 50 percent points of the gene expression intensity data obtained in channel c in grid k, respectively):
$$\begin{matrix}{w}_{i j}^{k} ( c ) = \frac{{y}_{i j}^{k} ( c ) - {L}_{k} ( c )}{{M}_{k} ( c ) - {L}_{k} ( c )} , \\ c = 1 , 2 , i = 1 , … , I , j = 1 , … , J , k = 1 , … , K .\end{matrix} \qquad ( 12 )$$

4. The cDNA microarray data correction system according to claim 1, wherein the order statistics are represented by the following EQ13 (where wᵢⱼ^{k}(c) is the standardized gene expression intensity data, yᵢⱼ^{k}(c) is gene expression intensity data of all spots obtained in a channel, and Ak(c), Lk(c) and Mk(c) indicate 35, 10 and 90 percent points of the gene expression intensity data obtained in channel c in grid k, respectively):
$$\begin{matrix}{w}_{i j}^{k} ( c ) = \frac{{y}_{i j}^{k} ( c ) - {A}_{k} ( c )}{{M}_{k} ( c ) - {L}_{k} ( c )} , \\ c = 1 , 2 , i = 1 , … , I , j = 1 , … , J , k = 1 , … , K .\end{matrix} \qquad ( 13 )$$

5. The cDNA microarray data correction system according to claim 3, wherein said data standardization means determines whether the gene expression intensity data of all spots obtained in at least two gene expression intensity data channels has been standardized and continues it until the gene expression intensity data of all spots has been standardized.

6. The cDNA microarray data correction system according to claim 1, wherein the standardized gene expression intensity data is represented by a sum of a true gene intensity and a distortion depending on the spot position.

7. The cDNA microarray data correction system according to claim 1, wherein said first correction means describes the distortion depending on the spot position by means of a nonparametric regression model represented by a regression relation of distortions with an x-axis, a y-axis, and an interaction of the x- and y-axes (αₖ^{(c)}(i),βₖ^{(c)}(j), and γₖ^{(c)}((i−mᵢ)(j−mⱼ)), respectively) and estimates the distortion depending on the spot position (ξᵢⱼ^{k}(c)) by the nonparametric smoothing method represented by the following EQ14:
\hat{ξ}ᵢⱼ^{k}(c)=\hat{α}ₖ^{(c)}(i)+\hat{β}ₖ^{(c)}(j)+\hat{γ}ₖ^{(c)}((i−mᵢ)(j−mⱼ)), c=1,2, i=1, . . . ,I, j=1, . . . ,J. (14)

8. The cDNA microarray data correction system according to claim 7, wherein the distortion depending on the spot position is corrected according to the following EQ15 (where \hat{z}ᵢⱼ^{k}(c) is corrected true gene expression intensity data):
\hat{z}ᵢⱼ^{k}(c)=wᵢⱼ^{k}(c)−\hat{ξ}ᵢⱼ^{k}(c) (15)

9. The cDNA microarray data correction system according to claim 8, wherein the S-D transformation in said second correction means is performed according to the following EQ16:
$$\begin{matrix}{u}_{i j}^{k} = {\hat{z}}_{i j}^{k} ( 1 ) + {\hat{z}}_{i j}^{k} ( 2 ) \\ {v}_{i j}^{k} = {\hat{z}}_{i j}^{k} ( 1 ) - {\hat{z}}_{i j}^{k} ( 2 )\end{matrix} \qquad ( 16 )$$

10. The cDNA microarray data correction system according to claim 9, wherein said second correction means describes the distortion by means of a nonparametric regression model represented by the following EQ17, estimates a measurement error caused by the difference in sensitivity between the fluorescent dyes by a nonparametric smoothing method represented by the following EQ18 and EQ19, and corrects the error:
vᵢⱼ^{k}=Φ(uᵢⱼ^{k})+εᵢⱼ^{k}, εᵢⱼ^{k}˜N(0, v²) (17)
ηᵢⱼ^{k}=vᵢⱼ^{k}−\hat{φ}(uᵢⱼ^{k}) (18)
$$\begin{matrix}{\hat{y}}_{i j}^{k} ( 1 ) = \frac{1}{2} ( {u}_{i j}^{k} + {η}_{i j}^{k} ) \\ {\hat{y}}_{i j}^{k} ( 2 ) = \frac{1}{2} ( {u}_{i j}^{k} - {η}_{i j}^{k} )\end{matrix} \qquad ( 19 )$$

11. The cDNA microarray data correction system according to claim 1, wherein, supposing that a probability of gene expression is lower than 0.5, it is assumed for the correction that the fluorescence intensity detected at more than half of the spots within each grid indicates a background noise or a systematic error.

12. The cDNA microarray data correction system according to claim 11, wherein, supposing that Lk(c) and Mk(c) indicate 25 and 50 percent points of the fluorescence intensity obtained in at least two gene expression intensity data channels in a grid, it is further assumed for the correction that Lk(c) and Mk(c)−Lk(c) are equal among the grids and teh channels on condition that most genes are in a non-expression state and that a distribution of 50 percent point or lower of the fluorescence intensity is common to all grids and channels.

13. A cDNA microarray data correction method of correcting global and local distortions of microarray data more precisely and correcting measurement errors caused by a difference in sensitivity between fluorescent dyes, comprising the steps of:
inputting previously-adjusted gene expression intensity data, considering flag information indicating a removal of background noise and reliability of each spot;
standardizing the gene expression intensity data by using grid-by-grid order statistics for the input gene expression intensity data on condition that most genes are in a non-expression state;
outputting the standardized gene expression intensity data;
estimating a distortion depending on the spot position on grid coordinates for the standardized gene expression intensity data by a nonparametric smoothing method and correcting the data distortion depending on the spot position;
outputting the first corrected gene expression intensity data whose distortion depending on the spot position has been corrected;
performing an S-D transformation for the first corrected gene expression intensity data, estimating a potential distortion caused by a difference in sensitivity between the fluorescent dyes in the gene expression intensity data by the nonparametric smoothing method, and correcting the distortion caused by the difference in sensitivity between the fluorescent dyes; and
outputting the second corrected gene expression intensity data whose distortion caused by the difference in sensitivity between the fluorescent dyes has been corrected.

14. The cDNA microarray data correction method according to claim 13, further comprising a step of quantifying the distortion of the gene expression intensity data in an arbitrary stage and visualizing it on an S-D plot.

15. The cDNA microarray data correction method according to claim 13, wherein the order statistics are represented by the following EQ20 (where wᵢⱼ^{k}(c) is the standardized gene expression intensity data, yᵢⱼ^{k}(c) is gene expression intensity data of all spots obtained in a channel, and Lk(c) and Mk(c) indicate 25 and 50 percent points of the gene expression intensity data obtained in channel c in grid k, respectively):
$$\begin{matrix}{w}_{i j}^{k} ( c ) = \frac{{y}_{i j}^{k} ( c ) - {L}_{k} ( c )}{{M}_{k} ( c ) - {L}_{k} ( c )} , \\ c = 1 , 2 , i = 1 , … , I , j = 1 , … , J , k = 1 , … , K .\end{matrix} \qquad ( 20 )$$

16. The cDNA microarray data correction method according to claim 13, wherein the order statistics are represented by the following EQ21 (where wᵢⱼ^{k}(c) is the standardized gene expression intensity data, yᵢⱼ^{k}(c) is gene expression intensity data of all spots obtained in a channel, and Ak(c), Lk(c) and Mk(c) indicate 35, 10 and 90 percent points of the gene expression intensity data obtained in channel c in grid k, respectively):
$${w}_{i j}^{k} ( c ) = \frac{{y}_{i j}^{k} ( c ) - {A}_{k} ( c )}{{M}_{k} ( c ) - {L}_{k} ( c )} , \\ c = 1 , 2 , i = 1 , … , I , j = 1 , … J , k = 1 , … , K . \qquad ( 21 )$$

17. The cDNA microarray data correction method according to claim 15, wherein, in the step of standardizing the data, it is determined whether the gene expression intensity data of all spots obtained in at least two gene expression intensity data channels have been standardized and it is continued until the gene expression intensity data of all spots have been standardized.

18. The cDNA microarray data correction method according to claim 17, wherein the standardized gene expression intensity data is represented by a sum of a true gene intensity and a distortion depending on the spot position.

19. The cDNA microarray data correction method according to claim 13, wherein, in the step of correcting the data distortion depending on the spot position, the distortion depending on the spot position is described by means of a nonparametric regression model represented by a regression relation of distortions with an x-axis, a y-axis, and an interaction of the x- and y-axes (αₖ^{(c)}(i),βₖ^{(c)}(j), and γₖ^{(c)}((i−mᵢ) (j−mᵢ)), respectively) and the distortion depending on the spot position (ξᵢⱼ^{k}(c)) is estimated by the nonparametric smoothing method represented by the following EQ22:
\hat{ξ}ᵢⱼ^{k}(c)=\hat{α}ₖ^{(c)}(i)+\hat{β}ₖ^{(c)}(j)+\hat{γ}ₖ^{(c)}(i−mᵢ) (j−mⱼ)), c=1,2, i=1, . . . , I, j=1, . . . , J. (22)

20. The cDNA microarray data correction method according to claim 19, wherein the distortion depending on the spot position is corrected according to the following EQ23 (where \hat{z}ᵢⱼ^{k}(c) is corrected true gene expression intensity data):
\hat{z}ᵢⱼ^{k}(c)=wᵢⱼ^{k}(c)−\hat{ξ}ᵢⱼ^{k}(c) (23)

21. The cDNA microarray data correction method according to claim 19, wherein the S-D transformation in the step of correcting the distortion caused by the difference in sensitivity between fluorescent dyes is performed according to the following EQ24:
$${u}_{i j}^{k} = {\hat{z}}_{i j}^{k} ( 1 ) + {\hat{z}}_{i j}^{k} ( 2 ) \\ {v}_{i j}^{k} = {\hat{z}}_{i j}^{k} ( 1 ) - {\hat{z}}_{i j}^{k} ( 2 ) \qquad ( 24 )$$

22. The cDNA microarray data correction method according to claim 20, wherein, in the step of correcting the distortion caused by the difference in sensitivity between the fluorescent dyes, the distortion is described by means of a nonparametric regression model represented by the following EQ25, a measurement error caused by the difference in sensitivity between the fluorescent dyes is estimated by a nonparametric smoothing method represented by the following EQ26 and EQ27, and the error is corrected:
vᵢⱼ^{k}=φ(uᵢⱼ^{k})+εᵢⱼ^{k}, εᵢⱼ^{k}=N(0, v²) (25)
ηᵢⱼ^{k}=vᵢⱼ^{k}−\hat{φ}(uᵢⱼ^{k}) (26)
$${\hat{y}}_{i j}^{k} ( 1 ) = \frac{1}{2} ( {u}_{i j}^{k} + {η}_{i j}^{k} ) \\ {\hat{y}}_{i j}^{k} ( 2 ) = \frac{1}{2} ( {u}_{i j}^{k} - {η}_{i j}^{k} ) \qquad ( 27 )$$

23. The cDNA microarray data correction method according to claim 13, wherein, supposing that a probability of gene expression is lower than 0.5, it is assumed for the correction that the fluorescence intensity detected at more than half of the spots within each grid indicates a background noise or a systematic error.

24. The cDNA microarray data correction method according to claim 23, wherein, supposing that Lk(c) and Mk(c) indicate 25 and 50 percent points of the fluorescence intensity obtained in at least two gene expression intensity data channels in a grid, it is further assumed for the correction that Lk(c) and Mk(c)−Lk(c) are equal among the grids and the channels on condition that most genes are in a non-expression state and that a distribution of 50 percent point or lower of the fluorescence intensity is common to all grids and channels.

25. The cDNA microarray data correction method according to claim 23, wherein, denoting that Ak(c), Lk(c) and Mk(c) indicate 35, 10 and 90 percent points of the fluorescence in a grid k for channel c, it is assumed for the correction that Ak(c) and Mk(c)−Lk(c) are common to all grids and channels.

26. A cDNA microarray data correction program for use in correcting global and local distortions of microarray data more precisely and correcting measurement errors caused by a difference in sensitivity between fluorescent dyes with a computer to execute the steps of:
inputting previously-adjusted gene expression intensity data, considering flag information indicating a removal of background noise and reliability of each spot;
standardizing the gene expression intensity data by using grid-by-grid order statistics for the input gene expression intensity data on condition that most genes are in a non-expression state;
outputting the standardized gene expression intensity data;
estimating a distortion depending on the spot position on grid coordinates for the standardized gene expression intensity data by a nonparametric smoothing method and correcting the data distortion depending on the spot position;
outputting the first corrected gene expression intensity data whose distortion depending on the spot position has been corrected;
performing an S-D transformation for the first corrected gene expression intensity data, estimating a potential distortion caused by a difference in sensitivity between the fluorescent dyes in the gene expression intensity data by the nonparametric smoothing method, and correcting the distortion caused by the difference in sensitivity between the fluorescent dyes; and
outputting the second corrected gene expression intensity data whose distortion caused by the difference in sensitivity between the fluorescent dyes has been corrected.

27. A computer-readable memory medium containing a cDNA microarray data correction program for use in correcting global and local distortions of microarray data more precisely and correcting measurement errors caused by a difference in sensitivity between fluorescent dyes with a computer to execute the steps of:
inputting previously-adjusted gene expression intensity data, considering flag information indicating a removal of background noise and reliability of each spot;
standardizing the gene expression intensity data by using grid-by-grid order statistics for the input gene expression intensity data on condition that most genes are in a non-expression state;
outputting the standardized gene expression intensity data;
estimating a distortion depending on the spot position on grid coordinates for the standardized gene expression intensity data by a nonparametric smoothing method and correcting the data distortion depending on the spot position;
outputting the first corrected gene expression intensity data whose distortion depending on the spot position has been corrected;
performing an S-D transformation for the first corrected gene expression intensity data, estimating a potential distortion caused by a difference in sensitivity between the fluorescent dyes in the gene expression intensity data by the nonparametric smoothing method, and correcting the distortion caused by the difference in sensitivity between the fluorescent dyes; and
outputting the second corrected gene expression intensity data whose distortion caused by the difference in sensitivity between the fluorescent dyes has been corrected.

28. The cDNA microarray data correction system according to claim 2, wherein the order statistics are represented by the following EQ12 (where wᵢⱼ^{k}(c) is the standardized gene expression intensity data, yᵢⱼ^{k}(c) is gene expression intensity data of all spots obtained in a channel, and Lk(c) and Mk(c) indicate 25 and 50 percent points of the gene expression intensity data obtained in channel c in grid k, respectively):
$${w}_{i j}^{k} ( c ) = \frac{{y}_{i j}^{k} ( c ) - {L}_{k} ( c )}{{M}_{k} ( c ) - {L}_{k} ( c )} , \\ c = 1 , 2 , i = 1 , … , I , j = 1 , … , J , k = 1 , … , K . \qquad ( 12 )$$

29. The cDNA microarray data correction system according to claim 2, wherein the order statistics are represented by the following EQ13 (where wᵢⱼ^{k}(c) is the standardized gene expression intensity data, yᵢⱼ^{k}(c) is gene expression intensity data of all spots obtained in a channel, and Ak(c), Lk(c) and Mk(c) indicate 35, 10 and 90 percent points of the gene expression intensity data obtained in channel c in grid k, respectively):
$${w}_{i j}^{k} ( c ) = \frac{{y}_{i j}^{k} ( c ) - {A}_{k} ( c )}{{M}_{k} ( c ) - {L}_{k} ( c )} , \\ c = 1 , 2 , i = 1 , … , I , j = 1 , … , J , k = 1 , … , K . \qquad ( 13 )$$

30. The cDNA microarray data correction system according to claim 4, wherein said data standardization means determines whether the gene expression intensity data of all spots obtained in at least two gene expression intensity data channels has been standardized and continues it until the gene expression intensity data of all spots has been standardized.

31. The cDNA microarray data correction method according to claim 14, wherein the order statistics are represented by the following EQ20 (where wᵢⱼ^{k}(c) is the standardized gene expression intensity data, yᵢⱼ^{k}(c) is gene expression intensity data of all spots obtained in a channel, and Lk(c) and Mk(c) indicate 25 and 50 percent points of the gene expression intensity data obtained in channel c in grid k, respectively):
$${w}_{i j}^{k} ( c ) = \frac{{y}_{i j}^{k} ( c ) - {L}_{k} ( c )}{{M}_{k} ( c ) - {L}_{k} ( c )} , \\ c = 1 , 2 , i = 1 , … , I , j = 1 , … , J , k = 1 , … , K . \qquad ( 20 )$$

32. The cDNA microarray data correction method according to claim 14, wherein the order statistics are represented by the following EQ21 (where wᵢⱼ^{k}(c) is the standardized gene expression intensity data, yᵢⱼ^{k}(c) is gene expression intensity data of all spots obtained in a channel, and Ak(c), Lk(c) and Mk(c) indicate 35, 10 and 90 percent points of the gene expression intensity data obtained in channel c in grid k, respectively):
$${w}_{i j}^{k} ( c ) = \frac{{y}_{i j}^{k} ( c ) - {A}_{k} ( c )}{{M}_{k} ( c ) - {L}_{k} ( c )} , \\ c = 1 , 2 , i = 1 , … , I , j = 1 , … J , k = 1 , … , K . \qquad ( 21 )$$

33. The cDNA microarray data correction method according to claim 16, wherein, in the step of standardizing the data, it is determined whether the gene expression intensity data of all spots obtained in at least two gene expression intensity data channels have been standardized and it is continued until the gene expression intensity data of all spots have been standardized.

34. The cDNA microarray data correction method according to claim 33, wherein the standardized gene expression intensity data is represented by a sum of a true gene intensity and a distortion depending on the spot position.
